(19) United States
(12) Patent Application Publication
(10) Pub. No.: US 2022/0175692 A1
(43) Pub. Date: Jun. 9, 2022

(54) PRESYNAPTIC GLUTAMATE RELEASE INHIBITORS FOR DECREASING NMDA ANTAGONIST SIDE EFFECTS IN ANESTHESIA

(71) Applicant: THE CLEVELAND CLINIC FOUNDATION, Cleveland, OH (US)

(72) Inventor: Amit Anand, Cleveland, OH (US)

(21) Appl. No.: 17/545,190

(22) Filed: Dec. 8, 2021

Related U.S. Application Data
(60) Provisional application No. 63/122,900, filed on Dec. 8, 2020.

(51) Int. Cl. A61K 31/02; A61K 31/4453; A61K 33/00; A61K 31/05; A61K 31/53; A61K 31/428; A61K 31/381; A61K 31/55; A61K 31/4166

(52) U.S. Cl. CPC A61K 31/02; A61K 31/4453; A61K 33/00; A61K 31/05; A61K 31/4166; A61K 31/428; A61K 31/381; A61K 31/55; A61K 31/53

(57) ABSTRACT

A method of anesthetizing a subject that includes administering a therapeutically effective amount of an NMDA antagonist to the subject is described. The method also includes administering an effective amount of a presynaptic glutamate release inhibitor to the subject to decrease the neurotoxic and/or psychomimetic side effects of the NMDA antagonist. Pharmaceutical compositions including an NMDA antagonist and a presynaptic glutamate release inhibitor are also described.

### CROSS-REFERENCE TO RELATED APPLICATIONS

This application claims priority to U.S. Provisional Application Ser. No. 63/122,900, filed on Dec. 8, 2020, which is hereby incorporated by reference in its entirety.

### BACKGROUND

Ketamine is an effective anesthetic that promotes hemodynamic stability and decreases postoperative opioid requirement, but also promotes psychological disturbances. Canet J., Castillo J., Anesthesiology, 116, 6-8 (2012). Despite the desirable analgesic and anti-inflammatory effects of ketamine, perioperative use has been limited because it promotes psychological disturbances. The incidence of psychologic disturbances associated with perioperative ketamine use reportedly ranges from 7% to 27%, presumably due to variation in amount of ketamine used and the use of diverse psychologic assessment methods and timeframes. Avidan et al., The Lancet, 390, 267-75 (2017).

How ketamine induces psychologic effects remains unclear, but glutamine release and modulation of the serotonergic system are plausible mechanisms. Lamotrigine, an anticonvulsant which decreases presynaptic glutamate release may thus reduce ketamine induced psychologic disturbances. Furthermore, lamotrigine augments serotonin reuptake inhibitors and may reduce ketamine-use disorder. Huang et al., Med. Hypotheses, 87, 97-100 (2016). For example, 300 mg oral lamotrigine significantly decreased ketamine-induced perceptual abnormalities in healthy subjects. It therefore seems likely that lamotrigine reduces the hyper-glutamatergic consequences of ketamine-induced NMDA receptor dysfunction. Moghaddam B., Adams B W, Science, 281, 1349-52 (1998).

NMDA antagonists such as ketamine can be used for anesthesia. However, their use has been limited due to side effects such as dissociation, delirium, and psychosis. Accordingly, there remains a need for a method of using NMDA antagonists for anesthesia while minimizing their undesirable side effects.

### SUMMARY OF THE INVENTION

In one aspect, the invention provides a method of anesthetizing a subject, comprising administering a therapeutically effective amount of an NMDA antagonist to the subject, the method further comprising administering an effective amount of a presynaptic glutamate release inhibitor to the subject to decrease the neurotoxic and/or psychomimetic side effects of the NMDA antagonist. In some embodiments, the NMDA antagonist is ketamine, while in further embodiments, the presynaptic glutamate release inhibitor is lamotrigine. In some embodiments, the presynaptic glutamate release inhibitor decreases the neurotoxic side effects of the NMDA antagonist, while in further embodiments, the presynaptic glutamate release inhibitor decreases the psychomimetic side effects of the NMDA antagonist.

The method can be used to treat a variety of different subjects in a variety of different situations. In some embodiments, the subject is being anesthetized perioperatively. In further embodiments, the subject is a pet or domestic animal. In additional embodiments, the subject is a human child or infant. In yet further embodiments, the anesthesia provided is general anesthesia.

Another aspect of the invention provides a pharmaceutical composition comprising a mixture of an NMDA antagonist and a presynaptic glutamate release inhibitor together with a pharmaceutically acceptable carrier. In some embodiments, the NMDA antagonist is ketamine, while in further embodiments, the presynaptic glutamate release inhibitor is lamotrigine. In additional embodiments, the pharmaceutical composition is a parenteral formulation.

### BRIEF DESCRIPTION OF THE FIGURES

FIG. 1 provides a graph showing the sample size needed to detect different relative risk assuming different reference proportions at power of 0.9. Points indicate sample size needed at each relative risk point on X axis for a given treatment effect (relative risk). The connecting lines only approximate the relationship for intermediate values.

FIG. 2 provides a table showing a summary of outcomes by treatment groups.

### DETAILED DESCRIPTION OF THE INVENTION

The present invention provides a method of anesthetizing a subject that includes administering a therapeutically effective amount of an NMDA antagonist to the subject. The method also includes administering an effective amount of a presynaptic glutamate release inhibitor to the subject to decrease the neurotoxic and/or psychomimetic side effects of the NMDA antagonist. The present invention also provides pharmaceutical compositions including an NMDA antagonist and a presynaptic glutamate release inhibitor.

### Definitions

The terminology as set forth herein is for description of the embodiments only and should not be construed as limiting of the invention as a whole. Unless otherwise specified, “a,” “an,” “the,” and “at least one” are used interchangeably. Furthermore, as used in the description of the invention and the appended claims, the singular forms “a”, “an”, and “the” are inclusive of their plural forms, unless contraindicated by the context surrounding such.

The terms “comprising” and variations thereof do not have a limiting meaning where these terms appear in the description and claims.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6,9, and 7.0 are explicitly contemplated.

“Treat”, “treating”, and “treatment”, etc., as used herein, refer to any action providing a benefit to a patient at risk for or afflicted with a disease, including improvement in the condition through lessening or suppression of at least one symptom, delay in progression of the disease, prevention or delay in the onset of the disease, etc.

“Pharmaceutically acceptable” as used herein means that the compound or composition is suitable for administration to a subject to achieve the treatments described herein, without unduly deleterious side effects in light of the severity of the disease and necessity of the treatment.

The terms “therapeutically effective” and “pharmacologically effective” are intended to qualify the amount of each agent which will achieve the goal of decreasing disease severity while avoiding adverse side effects such as those typically associated with alternative therapies. The therapeutically effective amount may be administered in one or more doses. An effective amount, on the other hand, is an amount sufficient to provide a significant chemical effect.

As used herein, “anesthesia” refers to loss of the ability to feel pain and a partial or complete loss of sensation, caused by administration of a drug or other medical intervention and is a local or general insensibility to pain with or without the loss of consciousness. “Epidural anesthesia” refers to that produced by injection of the anesthetic into the extradural space, either between the vertebral spines or into the sacral hiatus (caudal block). “General anesthesia” refers to a state of unconsciousness and insusceptibility to pain, produced by administration of anesthetic agents by, for example, inhalation, intravenously, intramuscularly, rectally, or via the gastrointestinal tract. “Spinal anesthesia” refers to a regional anesthesia by injection of a local anesthetic into the subarachnoid space around the spinal cord.

The term “anesthetic”, as used herein, refers to a drug or agent capable of producing a complete or partial loss of feeling (anesthesia).

An “antagonist” is a composition of matter which when administered to a mammal such as a human, inhibits a biological activity attributable to the level or presence of a compound or molecule of interest in the mammal.

The term “subject,” as used herein, refers to human or non-human mammal, e.g. a dog, a cat, a mouse, a rat, a cow, a sheep, a pig, a goat, or a primate, and expressly includes laboratory mammals, livestock, pets, and domestic mammals.

### Decreasing Side Effects in Anesthesia

One aspect of the invention provides a method of anesthetizing a subject. The method includes administering a therapeutically effective amount of an NMDA antagonist to the subject, the method further comprising administering an effective amount of a presynaptic glutamate release inhibitor to the subject to decrease the neurotoxic and/or psychomimetic side effects of the NMDA antagonist.

Anaesthesia is a state of controlled, temporary loss of sensation or awareness that is induced for medical purposes. It may include some or all of analgesia (relief from or prevention of pain), paralysis (muscle relaxation), amnesia (loss of memory), and unconsciousness. There are three categories of anesthesia. These are 1) general anesthesia, which suppresses central nervous system activity and results in unconsciousness and total lack of sensation, using either injected or inhaled drugs; 2) sedation, which suppresses the central nervous system to a lesser degree, inhibiting both anxiety and creation of long-term memories without resulting in unconsciousness; and regional and local anesthesia, which blocks transmission of nerve impulses from a specific part of the body.

In some embodiments, the anesthesia provided is general anesthesia. General anesthesia (as opposed to sedation or regional anesthesia) has three main goals: lack of movement (paralysis), unconsciousness, and blunting of the stress response. Anesthesia is typically provided through either inhaled or intravenous administration of the NMDA antagonist. The inhalational anesthetic delivery system used for inhaled anesthetics is an anesthetic machine. An anesthetic machine includes vaporizers, ventilators, an anesthetic breathing circuit, waste gas scavenging system and pressure gauges. Patients under general anesthesia must undergo continuous physiological monitoring to ensure safety. Monitoring can include electrocardiography (ECG), and monitoring of heart rate, blood pressure, inspired and expired gases, oxygen saturation of the blood (pulse oximetry), and temperature.

In one embodiment, the method is useful to provide anesthesia for surgery for periods of between 3 and 12 hours, and analgesia after surgery for at least 24 hours. When used to provide anesthesia for surgery, the subject is anesthetized perioperatively. The term “perioperative,” as used herein, refers to the three phases of surgery: preoperative, intraoperative, and postoperative. In some embodiments, the anesthesia is provided preoperatively, in some embodiments the anesthesia is provided intraoperatively, and in some embodiments the anesthesia is provided postoperatively.

In some embodiments, the subject is a human. In other embodiments, the subject is a pet or domestic animal. In another embodiment, the subject is a human infant. In another embodiment, the subject is a human child. In another embodiment, the subject is a young human child. In another embodiment, the subject is a human adult.

“Infant” refers, in another embodiment, to a subject under the age of 1 year. “Child” refers, in another embodiment, to a subject under the age of 18 years. “Young child” refers, in another embodiment, to a subject under the age of 7 years. “Adult” refers, in other embodiments, to a subject over 18. All of these age categories are used in the context of human subjects.

### NMDA Antagonists

NMDA receptor antagonists are a class of drugs that work to antagonize, or inhibit the action of, the N-Methyl-D-aspartate receptor. They are commonly used as anesthetics for animals and humans; the state of anesthesia they induce is referred to as dissociative anesthesia. Some NMDA receptor antagonists, such as ketamine, dextromethorphan (DXM), phencyclidine (PCP), methoxetamine (MXE), and nitrous oxide (N₂O), have dissociative, hallucinogenic, and euphoriant properties, which can be an undesirable side effect when the drugs are being used for anesthesia. Additional NMDA antagonists include chloroform, ethanol, propofol, MK-801, chlorphenidine, dephenidine, methoxyphenidine, and memantine. In some embodiments, the NMDA antagonist is ketamine, or its enantiomer esketamine.

Ketamine is one of the few NMDA antagonists that is currently being used in human medicine, but can have undesirable side effects. It was approved, for short-term use as a surgical anesthetic, roughly 40 years ago, before it was known to have any NMDA antagonist properties. However, despite the concerns over the neurotoxic side effects of NMDA antagonists, three factors apparently have convinced most surgeons and anesthesiologists that ketamine is sufficiently safe for normal use as a surgical anesthetic. First: as can be shown in cell culture tests, it is substantially less potent and aggressive, at binding to and blocking NMDA receptors, than MK-801 or phencyclidine. Second: it is used mainly for relatively brief periods, such as an hour or less, for surgeries such as setting a broken bone, or sewing up a wound (although in some cases it is still used for longer types of surgery). And third: anesthesiologists have realized that ketamine should be co-administered along with a benzodiazepine-type drug, such as diazepam, which helps suppress unwanted excessive neuronal activity. This co-administration of a sedative-type drug such as diazepam helps reduce the risk and severity of a transient form of disorientation and/or psychosis, commonly called a “ketamine emergence reaction”, which occurs among some surgery patients as they wake up after being treated with ketamine if administered without a second drug such as diazepam.

### Presynaptic Glutamate Release Inhibitors

An effective amount of a presynaptic glutamate release inhibitor can be administered to the subject to decrease the neurotoxic and/or psychomimetic side effects of the NMDA antagonist. A number of glutamate release inhibitors are known. See Obrenovitch, T. and Urenjak, J., Amino Acids, 14, 143-50 (1998). An additional family of disubstituted guanidines such as N-acenaphthyl-N′-methoxynaphthyl guanidine (CNS 1237) and its analogs have also been identified as being presynaptic glutamate release inhibitors. Goldin et al., Ann N Y Acad Sci, 765, 210-229 (1995). These agents suppress side-effect activity by inhibiting glutamate release. Examples of presynaptic glutamate release inhibitors include lamotrigine, riluzole, pomaglumetad, methionil, carbamazepine, and phenytoin.

In some embodiments, the presynaptic glutamate release inhibitor is lamotrigine. Lamotrigine is a member of the sodium channel blocking class of antiepileptic drugs. It may suppress the release of glutamate and aspartate, two dominant excitatory neurotransmitters in the central nervous system. Studies suggested that lamotrigine acts presynaptically on voltage-gated sodium channels to decrease glutamate release.

Several known drugs accomplish this by inhibiting the action of sodium ion channels controlled by “upstream” synaptic receptors, in a manner which reduces the number of “firing” events that neurons undergo over a span of time. Drugs which act by this mechanism include riluzole, lamotrigine, carbamazepine, and diphenylhydantoin. Riluzole has been reported to help relieve the symptoms of depression in at least some cases; accordingly, it offers a good candidate for inclusion as a safener drug in NMDA antagonist treatments designed to treat severe depression. Lamotrigine reportedly has a significant level of activity in relieving neuropathic pain; accordingly, it offers a good candidate for inclusion as a safener drug in NMDA antagonist treatments designed to treat neuropathic pain.

### Overcoming Side Effects of NMDA Antagonists

One of the disadvantages to using NMDA antagonists as anesthetics is that they can cause undesirable neurotoxic and/or psychomimetic side effects. The inventors have discovered that a presynaptic glutamate release inhibitor can be administered to the subject to decrease the neurotoxic and/or psychomimetic side effects of the NMDA antagonist. In some embodiments, the presynaptic glutamate release inhibitor decreases the neurotoxic side effects of the NMDA antagonist, while in additional embodiments the presynaptic glutamate release inhibitor decreases the psychomimetic side effects of the NMDA antagonist.

In the late 1980's and early 1990s, a number of NMDA antagonist were found to be effective. However, when these drugs were entered into human clinical trials, it became evident that NMDA antagonist drugs would induce hallucinations and other psychotomimetic effects, at doses which were lower than the dosages that were required to achieve any significant neuroprotective benefits. As various NMDA antagonist drugs have been developed and tested in human clinical trials, it has become increasingly evident that any drug which significantly blocks the functional activity of the NMDA receptor system (at least, at a level which offers a substantial potential for treating neurologic disorders such as described herein) will produce psychotomimetic side effects.

As used herein, the term “neurotoxic” implies that a drug can disrupt neuronal functioning to a point where neurons will begin dying, thereby leading to permanent brain damage. Accordingly, it is accurate to assert that potent NMDA antagonists such as dizocilpine and phencyclidine, and moderate (or moderately potent) NMDA antagonists such as ketamine, do indeed pose serious neurotoxic risks, and can cause neuronal death and permanent brain damage. However, as in all aspects of pharmacology and toxicology, it must be understood that any neurotoxic risks or effects of any NMDA antagonist drug will depend on the dosage and route of administration of the drug, and if prolonged or repeated administration of a drug is involved, any neurotoxic risks also will depend on the routes, duration(s), and timing of drug administration.

In the early days, when these types of side effects were displayed by patients anesthetized by PCP or ketamine, it was assumed that symptoms such as hallucinations were reversible, and reasonably benign, especially when ketamine was used for anesthesia. However, animal studies showed that, while the types of neuronal stresses imposed by a reasonably brief blockade of NMDA receptors appear to be reversible, a more prolonged blockade of NMDA receptors results in neuronal deaths, and permanent brain damage. Accordingly, researchers and regulatory authorities have been obliged to give very serious consideration to the evidence which indicates that: (i) psychotomimetic symptoms appear to function as warning signals, indicating that neurons are being placed under serious and possibly severe stress, whenever NMDA receptors are being blockaded at levels which begin triggering hallucinations and other psychotomimetic symptoms; and, (ii) if such stresses are prolonged, the affected neurons can be stressed so severely that they will die.

Available data indicate that there are strong correlations between: (i) the psychotomimetic symptoms caused by NMDA antagonist drugs, and (ii) the threat that permanent and serious brain damage will occur, if NMDA antagonist drugs are administered to patients suffering from unwanted NR/hyper activity, for prolonged periods of time and at dosages sufficient to achieve lasting therapeutic benefits. These data, along with various other concerns as described above, have posed major and severe obstacles to the use of NMDA antagonist drugs for treating neurologic or psychiatric disorders. Although limited and small-scale clinical trials are being done by scattered teams of researchers, commercial drug development and industry-supported research, in this particular field, apparently are at a standstill, and are not moving forward in any way which addresses or reflects the huge potential of such drugs to relieve suffering, and to treat or prevent a number of extremely severe medical problems.

### Pharmaceutical Mixtures

A pharmaceutical composition comprising a mixture of an NMDA antagonist and a presynaptic glutamate release inhibitor together with a pharmaceutically acceptable carrier. In some embodiments, the NMDA antagonist is selected from the group consisting of chloroform, ethanol, PCP, ketamine, nitrous oxide, and propofol. In further embodiments, the NMDA antagonist is ketamine. In yet further embodiments, the presynaptic glutamate release inhibitor is selected from the group consisting of lamotrigine, riluzole, pomaglumetad, methionil, carbamazepine, and phenytoin. In additional embodiments, the presynaptic glutamate release inhibitor is lamotrigine.

The pharmaceutical mixture can include any suitable dosages of the NMDA antagonist and presynaptic glutamate release inhibitor. The pharmaceutical mixture can also be provided in any of the formulations described herein. In some embodiments, the pharmaceutical composition is a parenteral formulation.

The compositions and dosage forms of the invention are useful for the administration of anesthetics for a variety of therapeutic purposes and procedures. Thus, for example, the compositions may be prepared for administration as blocks in advance of various surgical procedures, and for the treatment or prevention of pain, whether in advance of a surgical procedure or in treatment of a pre-existing condition; and more generally, for pain management, e.g. as part of a treatment regimen.

### Administration and Formulation

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

The pharmaceutical mixtures disclosed herein can be formulated and packaged in any of several well-known and conventional forms, provided that the dosage of each agent included in any such formulation must be clear and apparent (such as by an appropriate label on the package) to any physician who will decide on a preferred dosage regimen for each specific patient. For example, well-known orally-ingestible formulations include:

(1) tablets, in which a binder material is included with the active ingredients, to create a composition which, when compressed into the shape of a tablet that can be conveniently swallowed, will retain that shape, and which will dissolve after ingestion;

(2) capsules, in which a surrounding two-component capsule is used to enclose a heterogeneous material, which can be either a powdered or granular formulation, or a liquid or paste-type material;

(3) coated tablets, which can be used to protect a compressed tablet against stomach acids, and which can be designed to dissolve only after the coated tablet has passed through the stomach and has reached the small intestines.

Other formulations (such as a powder that must be added, in a controlled quantity, to an ingestible liquid, or an orally-ingestible liquid such as a syrup-type formulation) are also known, and can be created and used if desired; however, those types of orally-ingested formulations are not well suited for establishing the type of careful control, over a dosage regimen, that needs to be established for a mixture of neuroactive safener drugs such as disclosed herein. Instead, “unit dosage” formulations such as tablets or capsules are preferred for use as described herein.

The tablets, troches, pills, capsules, and the like may also contain one or more of the following: a binder such as gum tragacanth, acacia, corn starch or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it may further contain a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, sugar, and the like. A syrup or elixir may contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The active agent may be incorporated into sustained-release preparations and devices.

Formulations of the present invention suitable for oral administration may be presented as discrete units such as tablets, troches, capsules, lozenges, wafers, or cachets, each containing a predetermined amount of the active agent as a powder or granules, as liposomes containing the celecoxib derivatives, or as a solution or suspension in an aqueous liquor or non-aqueous liquid such as a syrup, an elixir, an emulsion, or a draught. For example, in one embodiment, the active agent is administered in drinking water. Such compositions and preparations typically contain at least about 0.1 wt-% of the active agent. The amount of the active agent is such that the dosage level will be effective to produce the desired result in the subject.

Since a sustained treatment regimen as disclosed herein, using a potent NMDA antagonist drug such as ketamine, should be carried out only in a hospital or clinic under conditions which allow continuous monitoring of a patient's vital signs, it is generally advisable to formulate a safener drug mixture, as disclosed herein, in orally-ingestible unit dosage formulations (such as tablets or capsules) that contain relatively low dosages, which typically will require at least two or more pills to be given to the patient each day. This “small dosage in each pill” approach can allow the care-giving staff (including physicians and nurses) to “fine tune” the dosage that is being given to any specific patient, by giving a suitable number of low-dosage tablets or capsules to a patient, during the course of each day, while adjusting the total daily dosage (i.e., the number of pills given to a patient during each day) in response to any effects that are reported by or observed in the patient.

Alternately, the pharmaceutical mixtures disclosed herein can be formulated in injectable form, such as in sealed sterile bags that render a liquid suited for continuous intravenous infusion. The use of adjustable clamps, placed on flexible infusion tubes in a manner that allows the “drip rate” for any specific patient to be controlled across a wide range of potential dosages, is a standard and well-known mode of administration for continuous infusion, and can be used for injectable liquids as disclosed herein. There is no need to administer a pharmaceutical mixture, as disclosed herein, via a separate tube that will be attached to its own distinct hypodermic needle; instead, an infusible safener drug mixture as disclosed herein can be added to the infusion bag which contains the NMDA antagonist drug (such as ketamine) that is being administered to the patient. If tests indicate that the mixing of the two distinct liquids, inside an infusion bag, must be promoted by active mixing, then any of various known mixing means can be used. For example, to avoid any direct (and potentially contaminating) contact between a mixing device and an infusion liquid, a pair of small external rollers can be provided which will travel in a repeating or reciprocate manner, along the length of the infusion bag, in a manner comparable to a peristaltic pump which drives fluid through a flexible tube by means of moving pinch rollers that squeeze the outside of the tube.

In some embodiments, the active agent is administered into the central nervous system. For example, the active agent may be administered into the cerebrospinal fluid. Special techniques are known for delivering therapeutic agents to the central nervous system. See Begley, Pharmacology & Therapeutics 104, 29-45 (2004), for a review of methods delivery of therapeutic agents to the central nervous system.

Any drug that is listed herein can be formulated as a “pharmacologically acceptable” salt or prodrug. Many of the neuroactive compounds disclosed herein tend to be mildly acidic or alkaline, and will dissociate when dissolved in an aqueous solution, in a manner which releases both (i) a neuroactive cation or anion, and (ii) a relatively unimportant single-atom anion or cation, which can be either a mineral (such as Na⁺, Cl⁻) or an organic ion (such as an acetate or maleate ion) which can be readily metabolized and used by the body. Accordingly, the use of salts to create “stabilized” formulations (often with increased and improved shelf-lives) of pharmaceutically active agents is a well-known practice, and can be used with the active agents discussed herein.

The term “prodrug” refers to a compound which is ingested or ingested in one form (which usually is inactive or only mildly active), which is then metabolized (usually by cleavage of a certain moiety from the initial compound, which will be catalyzed by one or more enzymes) inside the body, to create or release a fully active molecule. Frequently, prodrugs are used to create sustained-dosage formulations, since the metabolic “activation” of the active molecules which have entered the body will not occur immediately, and all at once. This approach has become well-known in the pharmaceutical arts, and can be used, if desired, to deliver any specific active agent of interest herein, if conditions indicate that administration of a prodrug, rather than a drug in an already-active form, is useful and advisable.

The pharmaceutical agents can be administered as pharmaceutically acceptable salts. Pharmaceutically acceptable salt refers to the relatively non-toxic, inorganic and organic acid addition salts of the pharmaceutical agents. These salts can be prepared in situ during the final isolation and purification of the compound, or by separately reacting a purified pharmaceutical agent with a suitable counterion, depending on the nature of the compound, and isolating the salt thus formed. Representative counterions include the chloride, bromide, nitrate, ammonium, sulfate, tosylate, phosphate, tartrate, ethylenediamine, and maleate salts, and the like. See for example Haynes et al., J. Pharm. Sci., 94, p. 2111-2120 (2005).

Other approaches also can be used to create dosage formulations, and in particular sustained dosage formulations, especially when orally-ingested formulations are involved. For example, an assortment of different binder materials and “microencapsulation” materials are known, which will dissolve at different rates, after ingestion. Accordingly, an encapsulated granular formulation can be created which will contain, for example, equal portions of three different types of granules, where the selection of three different binder or encapsulation materials will cause and enable ⅓ of the granules to dissolve rapidly, after being released by the outer capsule, while another ⅓ of the granules will dissolve more slowly, such as within 2 to 4 hours after being released from the outer capsule, and the final ⅓ of the granules will dissolve even more slowly, such as mainly within a span of about 4 to 8 hours after being released from the outer capsule.

With regard to liquid formulations that can be administered continuously via intravenous infusion, there is no particular need for the use of sustained-dosage formulations. Instead, the use of controlled infusion rates, throughout an infusion regimen which might last for days, can provide the desired results.

Finally, the treatments disclosed herein can be administered simultaneously or sequentially with various other treatments that: (i) have been used in the past to help a patient cope with a disease or disorder, or (ii) may have beneficial neuroprotective or other medical effects. These issues arise and will require attention because any patient who is seriously considering this type of treatment for a chronic condition will very likely be taking various medications and/or nutritional supplements in an effort to alleviate the problem that requires an intervention of the type described herein. Any such other treatments should be carefully evaluated by the treating physician, who will need to assess, for any individual patient, the likelihood of unwanted complications or potential benefits that might arise from either: (i) suspending, reducing, or otherwise altering the dosage of one or more other drugs or supplements to which the patient has become accustomed, habituated, or reliant upon; or, (ii) commencing any other pharmaceutical or other treatment which the physician believes may be helpful.

Thus, there has been shown and described a new and useful means for combining a presynaptic glutamate release inhibitor with an NMDA antagonist in ways which will allow such mixtures and combinations to prevent, reduce, and control the unwanted and potentially neurotoxic side effects of the NMDA antagonist drugs, more potently and effectively than was previously possible. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

The present invention is illustrated by the following example. It is to be understood that the particular examples, materials, amounts, and procedures are to be interpreted broadly in accordance with the scope and spirit of the invention as set forth herein.

### EXAMPLE

### Example 1: Lamotrigine for Reducing Ketamine-Induced Psychological Disturbances: A pilot Randomized and Blinded Trial

We report the finding of a pilot randomized and double-blind trial in which we tested the hypothesis that a single 300-mg dose of lamotrigine given a few hours before surgery reduces ketamine-induced psychological disturbances. We enrolled 46 adults (23 Lamotrigine, 23 placebo) 18-65 years who were scheduled for elective noncardiac surgery with general anesthesia with a planned overnight stay at Cleveland Clinic main campus between April 2019 and October 2019. All included patients provided written informed consent during their preoperative clinic visit. The trial was approved by Cleveland Clinic Institutional Review Board and was registered at clinicaltrails.gov [NCT03831854; PI Kamal Maheshwari; February 2019]. It was a priori defined as a pilot trial. We excluded patients who had a history of seizure and/or antiepileptic medication use, schizophrenia, unstable angina, or had previously taken lamotrigine.

All patients received ketamine 1 mg/kg bolus at induction of anesthesia followed by a 5-μg/kg/min infusion which was stopped at the end of surgery. Anesthesia generally included propofol, a muscle relaxant, fentanyl, and sevoflurane. The primary outcome was presence of the psychologic disturbances measured 30-90 min after admission to the post-anesthesia care unit. We evaluated four key items of the Brief Psychiatric Rating Scale corresponding to positive symptoms of schizophrenia: conceptual disorganization, hallucinatory behavior, suspiciousness, and unusual thought content. Each symptom was rated 1-7 (1=not present, 2=very mild, 3=mild, 4=moderate, 5=moderately severe, 6=severe, 7=extremely severe). Secondary outcomes were opioid use, pain, and postoperative nausea or vomiting in the post-anesthesia care unit.

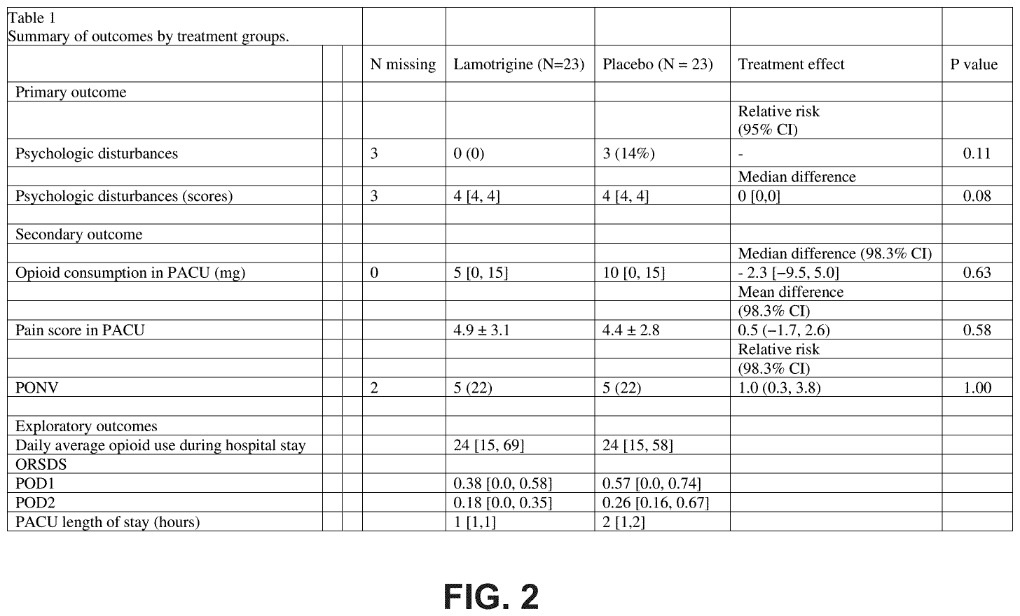

No patients randomized to lamotrigine had psychologic disturbances, whereas 3 (14%) assigned to placebo did. See FIG. 2. Psychological side effects were thus too sparse to justify formal statistical testing. Opioid use in morphine equivalents was a median of 5 [Q1, Q3: 0, 15] mg in patients randomized to lamotrigine and 10 [0, 15] mg in those assigned to placebo, with the median difference being −2.3 (98.3% CI; −9.5, 5.0, P=0.63). Mean±standard deviation pain scores were similar in the lamotrigine (4.9±3.1) and placebo (4.4±2.8) patients: difference in means of 0.5 (98.3% CI; −1.7, 2.6, P=0.58). Five patients in each group experienced nausea or vomiting.

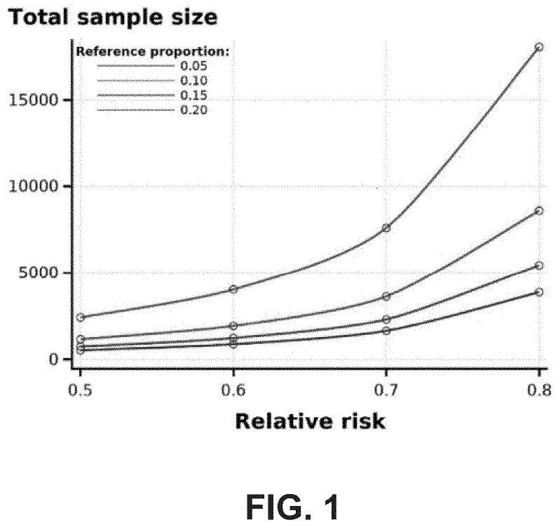

In this pilot randomized trial, despite giving a moderate dose of ketamine, only 6% of our patients displayed psychologic disturbances. Our estimate of the incidence of psychologic disturbances will inform perioperative ketamine use and future research. A large trial (FIG. 1) seems warranted to assess the potential ability of lamotrigine to prevent ketamine-induced psychologic disturbances.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of anesthetizing a subject, comprising administering a therapeutically effective amount of an NMDA antagonist to the subject, the method further comprising administering an effective amount of a presynaptic glutamate release inhibitor to the subject to decrease the neurotoxic and/or psychomimetic side effects of the NMDA antagonist.

2. The method of claim 1, wherein the NMDA antagonist is selected from the group consisting of chloroform, ethanol, PCP, ketamine, nitrous oxide, and propofol.

3. The method of claim 1, wherein the NMDA antagonist is ketamine.

4. The method of claim 1, wherein the presynaptic glutamate release inhibitor is selected from the group consisting of lamotrigine, riluzole, pomaglumetad, methionil, carbamazepine, and phenytoin.

5. The method of claim 1, wherein the presynaptic glutamate release inhibitor is lamotrigine.

6. The method of claim 1, wherein the subject is being anesthetized perioperatively.

7. The method of claim 1, wherein the subject is a pet or domestic animal.

8. The method of claim 1, wherein the subject is a human child or infant.

9. The method of claim 1, wherein the anesthesia is general anesthesia.

10. The method of claim 1, wherein the presynaptic glutamate release inhibitor decreases the neurotoxic side effects of the NMDA antagonist.

11. The method of claim 1, wherein the presynaptic glutamate release inhibitor decreases the psychomimetic side effects of the NMDA antagonist.

12. A pharmaceutical composition comprising a mixture of an NMDA antagonist and a presynaptic glutamate release inhibitor together with a pharmaceutically acceptable carrier.

14. The pharmaceutical composition of claim 12, wherein the NMDA antagonist is selected from the group consisting of chloroform, ethanol, PCP, ketamine, nitrous oxide, and propofol.

15. The pharmaceutical composition of claim 12, wherein the NMDA antagonist is ketamine.

16. The pharmaceutical composition of claim 12, wherein the presynaptic glutamate release inhibitor is selected from the group consisting of lamotrigine, riluzole, pomaglumetad, methionil, carbamazepine, and phenytoin.

17. The method of claim 1, wherein the presynaptic glutamate release inhibitor is lamotrigine.

18. The method of claim 1, wherein the pharmaceutical composition is a parenteral formulation.
